(19) United States
(12) Patent Application Publication
(10) Pub. No.: US 2009/0221864 A1
(43) Pub. Date: Sep. 3, 2009

(54) High Ethane Recovery Configurations And Methods In LNG Regasification Facility

(75) Inventor: John Mak, Santa Ana, CA (US)

Correspondence Address: FISH & ASSOCIATES, PC;ROBERT D. FISH, 2603 Main Street, Suite 1000, Irvine, CA, 92614-6232, US

(73) Assignee: FLUOR TECHNOLOGIES CORPORATION, Aliso Viejo, CA (US)

(21) Appl. No.: 12/299,164

(22) PCT Filed: May 23, 2007
(86) PCT No.: PCT/US07/12376

§ 371 Date: Apr. 27, 2009

Related U.S. Application Data
(60) Provisional application No. 60/808,091, filed on May 23, 2006.

(51) Int. Cl. C07C 7/00; B01J 8/04

(52) U.S. Cl. 585/803; 422/189

(57) ABSTRACT

LNG is processed in contemplated plants and methods such that refrigeration content of the LNG feed is used to provide reflux duty to the demethanizer and to further condense a vapor phase of the demethanizer overhead product. In such plants, the demethanizer provides a bottom product to a deethanizer, wherein a demethanizer side draw provides refrigeration to the deethanizer overhead product to thus form an ethane product and deethanizer reflux.

This application claims priority to our copending U.S. provisional patent application with the Ser. No. 60/808,091, which was filed May 23, 2006.

### FIELD OF THE INVENTION

The field of the invention is gas processing, especially as it relates to regasification of liquefied natural gas and/or recovery of C2, and C3 plus components.

### BACKGROUND OF THE INVENTION

While North American natural gas resources are depleting, the consumption of natural gas increases, mainly due to replacement of less efficient oil and coal fired power plants with more efficient and cleaner burning natural gas combined cycle power plants. The depletion of domestic natural gas also results in a reduction in Natural Gas Liquid (NGL) production, and therefore, import of liquefied natural gas (LNG) is considered crucial in North America.

In most foreign LNG export and liquefaction plants, removal of pentane, hexane, and heavier hydrocarbons is required to avoid wax formation in the cryogenic liquefaction exchanger. However, the ethane and LPG components (C2, and C3/C4+) are typically not removed and are liquefied together with the methane component, resulting in LNG with a fairly high gross heating value. Exemplary heating values of LNG from a number of LNG export plants in the Atlantic, Pacific Ocean and Middle East are shown in FIG. 1. The higher heating values indicate a higher proportion of the non-methane components, and the chemical composition (methane, ethane, propane, butane and heavier components) for such LNG is shown in FIG. 2.

In most import LNG, the ethane content typically ranges from about 4% to about 12% ethane, and the propane and heavier hydrocarbon content ranges from about 3% to about 6%. However, in at least some sources (see FIG. 2) significantly higher ethane, propane, and higher hydrocarbons are found. Thus, LNG import provides an attractive alternative source of ethane, propane and heavier hydrocarbons that can be extracted at the receiving terminals to meet industrial demands. However, most of the known processes for removal of NGL (i.e., C2, C3, and higher) do not effectively utilize the refrigeration content in LNG, and the ethane and propane recoveries of such processes are relatively low. For example, some processes operate by vaporizing the LNG in a flash drum and stripping the LNG in a demethanizer that operates at low pressures (the flash vapor and/or demethanizer overhead are then compressed to the pipeline pressure), while in other processes the demethanizer vapor is compressed to an intermediate pressure such that it can be re-condensed using inlet LNG as a coolant reducing compression power to some extent. An exemplary regasification process and configuration is described in U.S. Pat. No. 6,564,579 to McCartney. Unfortunately, such known processes are typically designed for ethane recovery of 50% ethane and propane recovery of 50% to 80%. Moreover, the vapor compression to meet the pipeline pressures or to achieve an intermediate pressure for re-condensation is often energy inefficient and costly.

A significantly more effective plant and method for LNG processing is described in our copending International patent application with serial number PCT/US05/22880 (WO 2006/004723), which is incorporated by reference herein. Here relatively high separation efficiency is achieved by utilizing LNG refrigeration content in a feed exchanger. In such plants, the demethanizer overhead is partially condensed using LNG cold and separated in a vapor phase and a liquid phase, wherein the liquid phase is used as demethanizer reflux and wherein the vapor phase is liquefied using the LNG cold. Once pumped to pipeline pressure, the liquefied vapor phase is then vaporized. However, while such configurations provide substantially improved energy efficiency and allow relatively high ethane recovery, ethane recoveries are still typically limited to 80%. Therefore, and especially where high ethane content is present in the import LNG and where even higher ethane recovery is desired, such plants are typically not suitable.

Consequently, while numerous processes and configurations for LNG regasification and NGL recovery are known in the art, all of almost suffer from one or more disadvantages. Most notably, many of the known NGL recovery processes require vapor compression, which is energy inefficient and has a generally low NGL recovery level. Moreover, known processes are also not suitable for high NGL recoveries (e.g., over 90% ethane and 99% propane) while producing 95% and better pure methane. Therefore, there is still a need to provide improved configurations and methods for NGL recovery in LNG regasification facilities.

### SUMMARY OF THE INVENTION

The present invention is directed to configurations and methods of LNG processing in which ethane and propane are recovered in an energy efficient manner at very high yields. In a typical configuration, ethane recovery is at least 90% and more typically 95% without the need for residue gas recompression. Propane plus recovery in such plants is typically 99% and higher. Among other parameters, such high efficiency and yield are due to the effective use of refrigeration content of the LNG in a feed exchanger and in a side reboiler/side draw that provides cold to the deethanizer overhead and demethanizer reflux.

In one aspect of the inventive subject matter, an LNG processing plant has a refluxed demethanizer that is fluidly coupled to a refluxed deethanizer such that the demethanizer provides a bottom product to the deethanizer. A heat exchange circuit is then coupled to the demethanizer and configured to use a side draw of the demethanizer to condense the deethanizer overhead product to thereby provide a reflux stream to the deethanizer and an ethane liquid. A feed exchanger is fluidly coupled to the refluxed demethanizer and is further configured to provide refrigeration to the demethanizer overhead product and the vapor portion of the demethanizer overhead product in an amount sufficient to liquefy the vapor portion of the demethanizer overhead product.

Viewed from a different perspective, a method of LNG processing will therefore include a step of providing a bottom product from a refluxed demethanizer to a refluxed deethanizer, and a further step of using a side draw of the demethanizer in a heat exchange circuit to condense a deethanizer overhead product to thereby form a reflux stream to the deethanizer and an ethane liquid. In yet another step, refrigeration is provided in a feed exchanger to a demethanizer overhead product and a vapor portion of the demethanizer overhead product in an amount sufficient to liquefy the vapor portion of the demethanizer overhead product.

Most preferably, the heat exchange circuit comprises a demethanizer side reboiler that provides refrigeration content to the deethanizer overhead product to thereby liquefy the deethanizer overhead product. In such configurations, a surge drum is typically configured to receive the liquefied deethanizer overhead product and is further typically configured to provide at least some of the liquefied deethanizer overhead product to the deethanizer as the reflux stream. Alternatively, the heat exchange circuit may also comprise an integral coil in the deethanizer head, wherein the coil receives a side draw from the demethanizer to thereby provide refrigeration content to the deethanizer overhead product to thus liquefy the deethanizer overhead product. Regardless of the nature of the circuit, it is preferred that the heat exchange circuit is configured such that the deethanizer overhead temperature is between −25° F. and −35° F.

With respect to the deethanizer it is preferred that the deethanizer is configured to operate at a pressure of between 80 psig and 150 psig and/or at an overhead temperature between −25° F. and −35° F. In most plants, a separator is included that separates the demethanizer overhead product into the vapor portion and a liquid portion, wherein the separator is fluidly coupled to the demethanizer such that the liquid portion is fed to the demethanizer as a demethanizer reflux stream. Typically, a pump is fluidly coupled to the feed exchanger to pump the liquefied vapor portion of the demethanizer overhead product to pipeline pressure, and the feed exchanger and the heat exchange circuit are configured to allow ethane recovery of at least 95% and methane purity of at least 99%.

Various objects, features, aspects and advantages of the present invention will become more apparent from the following detailed description of preferred embodiments of the invention.

### BRIEF DESCRIPTION OF THE DRAWING

FIG. 1 is a schematic illustration of heating values of LNG from various export plants in the Atlantic, Pacific, and Middle East.

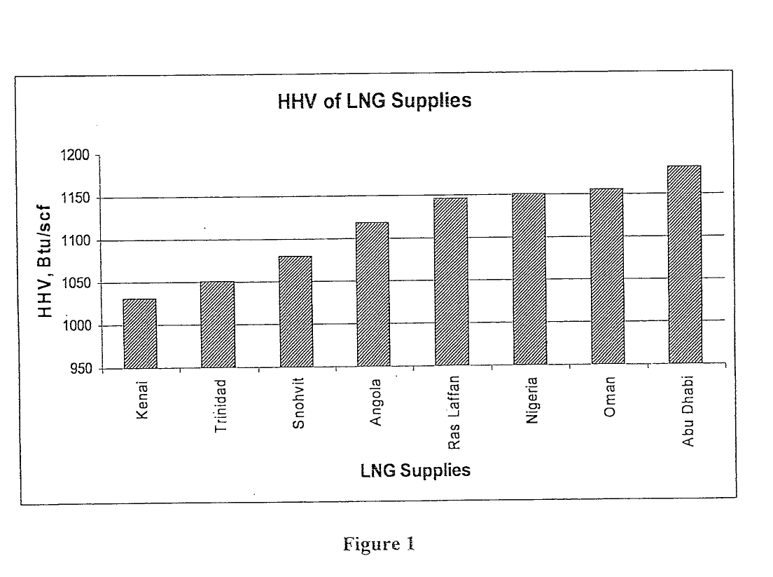

FIG. 2 is a schematic illustration of the chemical composition of LNG from the sources of FIG. 1.

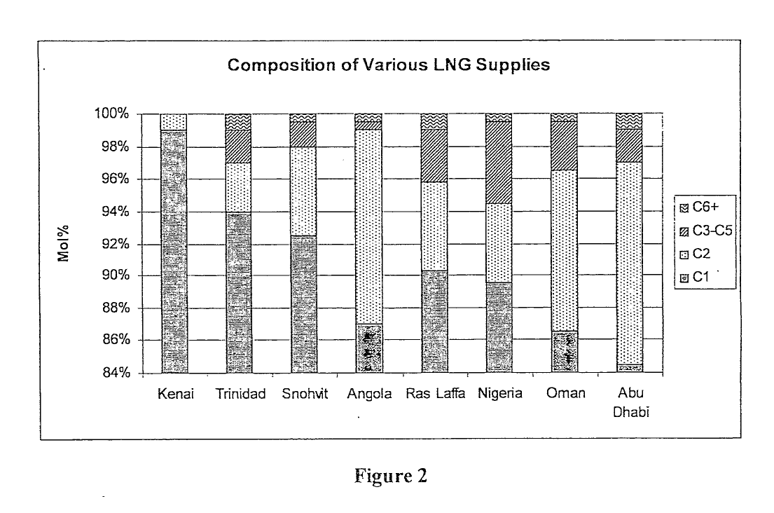

FIG. 3 is an exemplary schematic illustration of an LNG processing plant according to the inventive subject matter.

FIG. 4 is a graph showing composite curves of the feed gas exchanger and the deethanizer reflux exchanger of FIG. 3

FIG. 5 is an exemplary schematic illustration of a further LNG processing plant according to the inventive subject matter.

### DETAILED DESCRIPTION

The present invention is directed to configurations and methods of processing LNG in which about 95% of the ethane and about 99% of the propane are recovered from (typically import) LNG producing a processed LNG with over 99% methane. The so formed processed LNG may then be further pressurized and regasified to the sales gas pipeline. Preferably, the processing of the LNG is performed in a refluxed demethanizer, using LNG cold for cooling. Processing still further preferably includes a refluxed deethanizer that uses the demethanizer side reboiler duty for refluxing the deethanizer.

Therefore, it should be recognized that LNG can be processed in a manner that takes full advantage of the cryogenic portion (i.e. −250° F. to −140° F.) of refrigeration content in the import LNG. More specifically, the inventor has discovered that an LNG stream can be pumped to a desired pressure and then used to supply both, reflux cooling in a demethanizer, and re-liquefaction of the demethanizer reflux drum vapor, while a demethanizer side reboiler is employed to supply reflux to the deethanizer. Most typically, and viewed from a different perspective, the pumped LNG stream is processed in the demethanizer to thereby form the streams that are cooled by the pumped LNG. Such configurations can deliver a processed lean LNG with 99% methane purity, while recovering at least 95% ethane and at least 99% propane from import LNG as products.

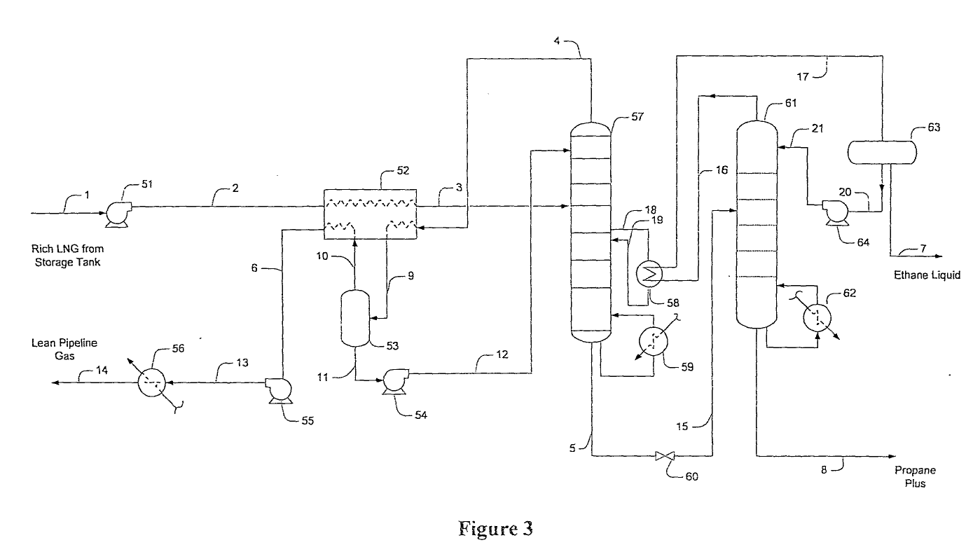

More specifically, and with further reference to the exemplary plant of FIG. 3, the LNG flow rate to the plant is equivalent to 2,000 MMscfd of natural gas. Rich LNG stream 1, with a typical gas composition shown in Table 1 below (unless indicated otherwise, all numbers in the table are expressed as mol fraction), is provided from a storage tank or vapor re-condenser (or other suitable source) at a pressure of about 80 to 100 psia or higher and a temperature of about −250° F. Stream 1 is pumped by LNG pump 51 to a suitable pressure, typically at about 300-350 psig to about 750 psig (even higher pressures of up to 1500 psig and in some cases above 1500 psig may be employed where a power-producing configuration is employed) forming stream 2, which is heated and partially vaporized in exchanger 52 by heat exchange with the demethanizer overhead stream 4 and reflux drum vapor stream 10. The exchanger outlet stream 3 at about −125° F. to −145° F. is fed to the upper section of the demethanizer 57. The demethanizer 57 produces the lean overhead vapor 4, typically with 97% to 99% methane purity, and recovers 95% of the ethane and over 99% of the propane content from the import LNG.

| | Rich LNG | Press. | LNG from | DeC1 | DeC1 | Lean | Ethane | Propane |
|---|---|---|---|---|---|---|---|---|
| | Feed | LNG | 52 | Ovhd | Bottoms | LNG | Product | Plus |
| Stream | 1 | 2 | 3 | 4 | 5 | 6 | 7 | 8 |
| Number | | | | | | | | |
| Nitrogen | 0.0017 | 0.0017 | 0.0017 | 0.0020 | 0.0000 | 0.0020 | 0.0000 | 0.0000 |
| Methane | 0.8598 | 0.8598 | 0.8598 | 0.9926 | 0.0091 | 0.9926 | 0.0144 | 0.0000 |
| Ethane | 0.0869 | 0.0869 | 0.0869 | 0.0054 | 0.6085 | 0.0054 | 0.9526 | 0.0100 |
| Propane | 0.0347 | 0.0347 | 0.0347 | 0.0000 | 0.2571 | 0.0000 | 0.0330 | 0.6469 |
| i-Butane | 0.0085 | 0.0085 | 0.0085 | 0.0000 | 0.0630 | 0.0000 | 0.0001 | 0.1725 |
| n-Butane | 0.0079 | 0.0079 | 0.0079 | 0.0000 | 0.0584 | 0.0000 | 0.0000 | 0.1600 |
| n-Pentane | 0.0005 | 0.0005 | 0.0005 | 0.0000 | 0.0039 | 0.0000 | 0.0000 | 0.0105 |
| Std Gas Flow | 2,000 | 2,000 | 2,000 | 1,730 | 270 | 1,730 | 172 | 99 |
| [MMSCFD] | | | | | | | | |
| Std Ideal | 875,523 | 875,523 | 875,523 | 698,653 | 176,870 | 698,653 | 108,550 | 68,320 |
| Liq Vol Flow | | | | | | | | |
| [barrel/day] | | | | | | | | |
| Temperature | −252 | −249 | −133 | −132 | 102 | −136 | −54 | 79 |
| [° F.] | | | | | | | | |
| Pressure | 103 | 550 | 540 | 495 | 500 | 480 | 100 | 110 |
| [psia] | | | | | | | | |

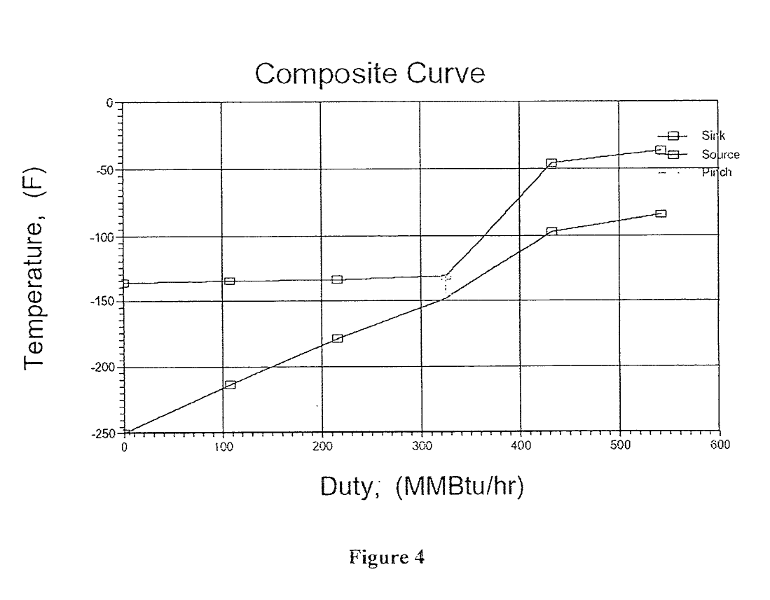

Demethanizer 57 typically operates at 450 psig to 550 psig. The pressure is adjusted according to the import LNG compositions and generally increases with the heating values of the import LNG to avoid temperature pinch in the feed chiller 52 (See FIG. 4). It should be especially noted that side reboiler 58 is used to supply reflux cooling to the deethanizer 61 by withdrawing a side stream 18 from lower section of the demethanizer, and by using heat from deethanizer overhead stream 16 to thus form heated stream 19. The demethanizer bottom composition is controlled by temperature of stream 5, at about 80° F. to 120° F., using bottom reboiler 59. Thus, it should be especially appreciated that in most aspects of contemplated configurations the set point of the demethanizer bottom temperature will increase with the ethane and propane content of import LNG to achieve 95% ethane recovery and 99% propane recovery while maintaining a low methane content (typically less than 1%) in the bottoms product. Demethanizer bottom product 5 is let down in pressure forming stream 15 using valve 60 to about 100 to 250 psig to feed the mid section of the deethanizer 61.

It should be appreciated that with the use of the demethanizer side reboiler cooling, the deethanizer can operate at a pressure of between about 200 psig to about 300 psig, more preferably at between 100 psig and 200 psig, and most preferably at between about 80 psig to 150 psig (e.g., at about 100 psig), which is significantly lower than conventional deethanizer operation (typically at about 350 psig). The lower pressure is advantageous from an energy cost aspect as the relative volatility between ethane and propane increases at the lower pressures making easier separation. With the use of the demethanizer side reboiler (at about −50° F. to −80° F.), the deethanizer overhead temperature can be lowered to about 40° F. to −20° F., and more typically -30° F.+/−5° F., which allows reduction of the deethanizer operating pressure, typically to 100 psig. The lower deethanizer pressure consequently requires less fractionation trays and less reboiler duty as the fractionation efficiency improves at the lower pressure.

The deethanizer overhead stream 61 is typically totally condensed at about −30° F. to −10° F. utilizing the refrigeration release from the demethanizer side reboiler 58. Deethanizer overhead condensed stream 17 is stored in surge drum 63. A portion (stream 20) is pumped by reflux pump 64 forming stream 21 as deethanizer reflux. Another portion (stream 7) is withdrawn as liquefied ethane product. The deethanizer 61 also produces a bottom product stream 8 with heat supplied by reboiler 62 (e.g., using a glycol heat transfer system as heat source).

The demethanizer overhead 4, which is typically at a pressure of about 350 psig to 550 psig and a temperature of at about −125° F. to −145° F. is cooled and partially condensed in exchanger 52 at a temperature of about −130° F. to −145° F. The so generated two-phase stream 9 is then separated in separator 53 into a liquid stream 11 containing over 95% methane and a lean vapor stream 10 containing over 99% methane. Liquid stream 11 is pumped by reflux pump 54 and returned to the top of the demethanizer 57 as a cold lean reflux stream 12. The separator vapor stream 10 is further cooled and condensed in exchanger 52 forming stream 6.

It should be especially recognized that overhead exchanger 52 provides two functions, providing reflux to the demethanizer to achieve a high ethane and propane recovery, and to condense the separator vapor to a liquid that allows the liquid to be pumped (rather than vapor compression), thus substantially lowering energy consumption, capital, and operational costs. The lean liquid stream 6, typically at a temperature of about −130° to about −145° F. is pumped by pump 55 to about 1000 psig to 1500 psig, as necessary for pipeline transmission pressure. The pressurized lean LNG stream 13 is further heated in vaporizer 56 forming stream 14 which is at about 50° F., or other temperature needed to meet pipeline requirements. It should be noted that suitable heat sources for the exchangers 59, 62, and 56 include all known heat sources (e.g., direct heat sources such as fired heaters, seawater exchangers, etc., or indirect heat sources such as glycol heat transfer systems). Typical gas compositions, flows temperatures, and pressures of the key process streams are shown in Table 1. Of course, it should be appreciated that for other feed compositions the heat and material balance would be slightly different. However, it should be noted that even for significantly altered gas compositions, the configurations and/or advantages of the inventive subject matter still remain.

The high efficiency of the fractionation process can be appreciated in the composite curves of the feed gas exchanger 52 and the deethanizer reflux exchanger 58 as depicted in FIG. 4. It should be noted that the heat sink and heat source curves are very closely matched with the temperature pinch occurring at the condensation of the demethanizer overhead in generating reflux (the pressure of the demethanizer will typically have to be adjusted between 450 psig to 650 psig according to avoid this pinch). In this process, over 50% of the cooling duty by LNG is used in re-liquefaction of the residue gas from the demethanizer reflux drum overhead vapor.

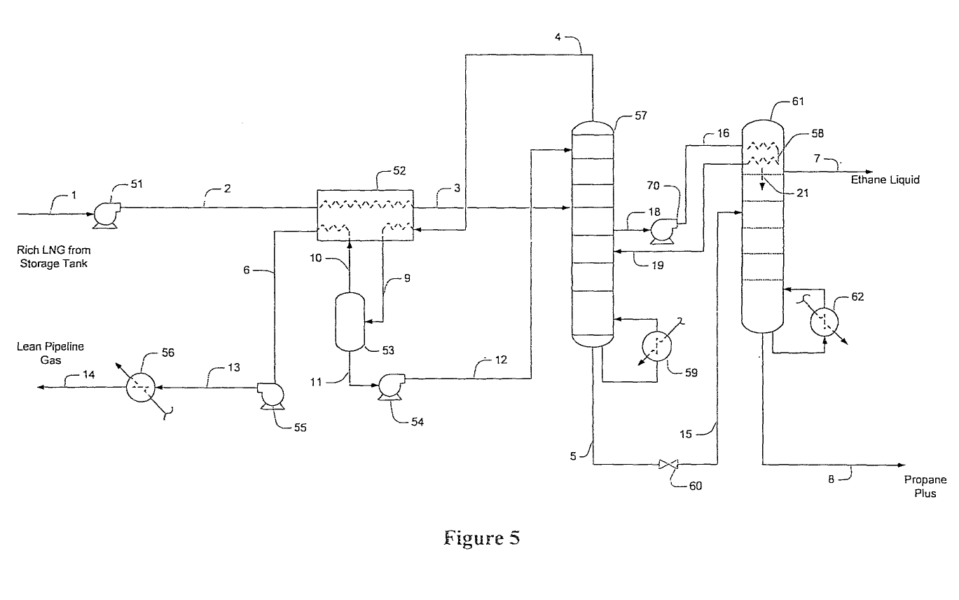

Alternatively, the demethanizer side reboiler 58 can be configured as an integral coil on top of the deethanizer 61, as shown in the schematic view of a second exemplary plant of FIG. 5. In this configuration, stream 18 is withdrawn from the lower section of the demethanizer 57, pumped by pump 70 to provide stream 16 for cooling in reflux exchanger 58 that is integral to the top of the deethanizer overhead column. Heated stream 19 is returned to the demethanizer. This provides an internal reflux stream 21, and the ethane product is drawn from the overhead system as stream 7. The front section of the plant is identical to the configuration of FIG. 3 and with respect to the remaining numerals of the components of FIG. 5, it should be noted that like components of FIG. 5 have same numerals in FIG. 3.

Thus, in preferred aspects of the inventive subject matter, the LNG processing plant has a heat exchanger that is configured such that at least part of the refrigeration content of import LNG passing through the exchanger provides refrigeration to a demethanizer reflux stream and further provides condensation refrigeration to a demethanizer reflux drum overhead product. Most typically, the LNG passing through the exchanger has a pressure of between 300 psig to 600 psig. A pump may further be coupled to the exchanger that pumps the condensed demethanizer reflux drum overhead to sales gas pipeline gas pressure. Preferred absorber feed pressures are between about 450 psig and 750 psig, while separation pressures are preferably between about 400 psig and 600 psig, and sales gas delivery pressures are preferably between about 700 psig and 1300 psig or higher. Consequently, the inventors contemplate a method of processing LNG in which LNG is provided and pumped to an absorber feed pressure. In especially contemplated ethane recovery plants where over 95% ethane recovery is desirable, the demethanizer bottoms can be further processed in a deethanizer column to produce a C2 overhead liquid, and a C3+ bottoms product. In this case, the deethanizer overhead reflux duty can be supplied by the side reboiler duty in the demethanizer in an external reflux system or integral reflux exchanger.

Therefore, it should be recognized that numerous advantages may be achieved using configurations according to the inventive subject matter. Among other things, it should be appreciated that contemplated configurations can recover over 95% of ethane and over 99% of propane from the import LNG, producing a processed LNG containing over 99% methane. This process allows processing of import LNG with varying compositions and heat contents while producing a 99% methane natural gas that can be used for pipeline gas and LNG transportation fuel for the North American market or other emission sensitive markets. Moreover, contemplated configurations will produce high-purity LPG liquid fuel, butane plus for gasoline blending and ethane as petrochemical feedstock or as energy source for the combined cycle power plant.

Further suitable contemplations and configurations are described in our copending International patent application with serial number PCT/US05/22880 (published as WO 2006/004723), which was filed Jun. 27, 2005, and which is incorporated by reference herein. For example, where power is to be extracted from the compressed feed gas, configurations are contemplated in which the liquid portion of the feed is pumped to pressure and heated to form a heated compressed liquid that is then expanded in a turbine to produce power. The so expanded stream is then fed to the demethanizer as before.

Thus, specific embodiments and applications of LNG processing and regasification configurations and methods have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. An LNG processing plant comprising:
a refluxed demethanizer that is fluidly coupled to a refluxed deethanizer such that the demethanizer provides a bottom product to the deethanizer;
a heat exchange circuit that is coupled to the demethanizer and that is configured to use a side draw of the demethanizer to condense a deethanizer overhead product to thereby provide a reflux stream to the deethanizer and an ethane liquid; and
a feed exchanger that is fluidly coupled to the refluxed demethanizer and that is further configured to provide refrigeration to a demethanizer overhead product and a vapor portion of the demethanizer overhead product in an amount sufficient to liquefy the vapor portion of the demethanizer overhead product.

2. The LNG processing plant of claim 1 wherein the heat exchange circuit comprises a demethanizer side reboiler that provides refrigeration content to the deethanizer overhead product to thereby liquefy the deethanizer overhead product.

3. The LNG processing plant of claim 2 further comprising a surge drum configured to receive the liquefied deethanizer overhead product and further configured to provide at least some of the liquefied deethanizer overhead product to the deethanizer as the reflux stream.

4. The LNG processing plant of claim 1 wherein the heat exchange circuit comprises an integral coil in the deethanizer head, and wherein the coil is configured to receive a side draw from the demethanizer to thereby provide refrigeration content to the deethanizer overhead product to thereby liquefy the deethanizer overhead product.

5. The LNG processing plant of claim 1 wherein the heat exchange circuit is configured such that the deethanizer overhead temperature is between −25° F. and −35° F.

6. The LNG processing plant of claim 1 wherein the deethanizer is configured to operate at a pressure of between 80 psig and 150 psig.

7. The LNG processing plant of claim 1 wherein a separator separates the demethanizer overhead product into the vapor portion and a liquid portion, and wherein the separator is fluidly coupled to the demethanizer such that the liquid portion is fed to the demethanizer as a demethanizer reflux stream.

8. The LNG processing plant of claim 1 further comprising a pump that is fluidly coupled to the feed exchanger to pump the liquefied vapor portion of the demethanizer overhead product to pipeline pressure.

9. The LNG processing plant of claim 1 wherein the feed exchanger and the heat exchange circuit are configured to allow ethane recovery of at least 95% and methane purity of at least 99%.

10. The LNG processing plant of claim 1 further comprising a pump that pumps LNG to the feed exchanger at a pressure of 300 psig to 1500 psig.

11. A method of LNG processing comprising:
providing a bottom product from a refluxed demethanizer to a refluxed deethanizer;
using a side draw of the demethanizer in a heat exchange circuit to condense a deethanizer overhead product to thereby form a reflux stream to the deethanizer and an ethane liquid; and
providing in an LNG feed exchanger refrigeration to a demethanizer overhead product and a vapor portion of the demethanizer overhead product in an amount sufficient to liquefy the vapor portion of the demethanizer overhead product.

12. The method of claim 11 wherein the heat exchange circuit comprises a demethanizer side reboiler that provides refrigeration content to the deethanizer overhead product to thereby liquefy the deethanizer overhead product.

13. The method of claim 12 wherein a portion of the liquefied deethanizer overhead product is fed to the deethanizer as the reflux stream.

14. The method of claim 11 wherein the heat exchange circuit comprises an integral coil in the deethanizer head, and wherein the coil receives a side draw from the demethanizer to thereby provide refrigeration content to the deethanizer overhead product to thereby liquefy the deethanizer overhead product.

15. The method of claim 11 wherein the deethanizer is operated at an overhead temperature between −25° F. and −35° F.

16. The method of claim 11 wherein the deethanizer is operated at a pressure of between 80 psig and 150 psig.

17. The method of claim 11 further comprising a step of separating the demethanizer overhead product into the vapor portion and a liquid portion, and feeding the liquid portion to the demethanizer as a demethanizer reflux stream.

18. The method of claim 11 further comprising a step of pumping the liquefied vapor portion of the demethanizer overhead product to pipeline pressure.

19. The method of claim 11 wherein the feed exchanger and the heat exchange circuit are configured to allow ethane recovery of at least 95% and methane purity of at least 99%.

20. The method of claim 11 further comprising a step of pumping LNG to the feed exchanger at a pressure of 300 psig to 1500 psig.
